(19) United States
(12) Patent Application Publication
(10) Pub. No.: US 2023/0338175 A1
(43) Pub. Date: Oct. 26, 2023

(54) REPOSITIONABLE INTRACRANIAL STENT WITH RETRIEVAL MECHANISM

(71) Applicant: ACCUMEDICAL BEIJING LTD, Beijing (CN)

(72) Inventors: Jin Park, Morris Plains, NJ (US); Daniel Olsen, Califon, NJ (US); Cory Zegel, Hoboken, NJ (US)

(73) Assignee: ACCUMEDICAL BEIJING LTD., Beijing (CN)

(21) Appl. No.: 17/729,842

(22) Filed: Apr. 26, 2022

(51) Int. Cl. A61F 2/966; A61F 2/844; A61F 2/90

(52) U.S. Cl. CPC A61F 2/966; A61F 2/844; A61F 2/90

(57) ABSTRACT

A stent device is described. The stent device includes a stent and a stent delivery system. The stent includes a cylindrical portion forming a stent frame having an outer lattice network of a plurality of interconnecting segments configured to exert a radial force against an inner wall of a blood vessel. The stent delivery system includes a locking mechanism configured to engage with the proximal end of the stent frame when covered in a sheath, and a push wire. The locking mechanism includes a proximal retention disk and a distal retention disk, the distal retention disk having a plurality of slots configured to engage with an equal plurality of struts formed on a proximal end of the stent.

### TECHNICAL FIELD

The present invention is generally related to the field of intravascular therapeutic devices and delivery systems, and more specifically to expandable stents and their corresponding delivery systems. This invention is particularly related to very small expandable stents and delivery systems for use in the neurovascular anatomy or similar vascular anatomies for treatment of occluded blood vessels, aneurysms, or other blood vessel disorders.

### BACKGROUND

Blood vessel disorders, specifically those affecting the neurovasculature, including intracranial atherosclerotic disease (ICAD) and aneurysms are a significant point of interest for innovation. Per the Center for Disease Control and Prevention (CDC), in 2020, cerebrovascular diseases were the fifth highest cause of deaths of any cause in the United States.

Stents and similar stenting technology have been successfully used in larger blood vessels in the cardiovascular and peripheral spaces. However, stents and stenting technology that has been used in larger blood vessels, such as the coronary and peripheral anatomy, and has been leveraged for smaller neurovasculature, encounters additional complications due to the smaller diameter of blood vessels in the neurovasculature.

Stenting involves entering a patient's vascular system and deploying a mesh tube to provide structural support to a blood vessel. This may be done to treat blood vessels that have fatty deposits or plaque built up in the blood vessel, otherwise known as atherosclerosis, or to support a vessel that was damaged or whose structural integrity is in question. In the neurovasculature, stenting is most notably used to prevent strokes or treat recurring strokes.

Stents may either be self-expanding or expanded via a mechanical method such as a balloon. As manufactured, stents are loaded onto an appropriate delivery system, either inside of an introducer sheath for a self-expanding stent, or onto a balloon catheter for a non-self-expanding stent. Procedurally, the interventionalist will locate the treatment site using a combination of accessories, typically including introducers, guide catheters, guide wires, microcatheters, and fluoroscopic imaging techniques. The interventionalist will then insert the delivery system into a guide catheter or microcatheter appropriately sized for the anatomy being treated. The delivery system with the pre-loaded stent will then be advanced to the target location and deployed by either retracting the guide catheter or microcatheter for self-expanding stents or by applying internal pressure to a balloon catheter mechanically forcing the stent to open for a non-self-expanding stent.

Stenting in small and tortuous vessels, such as the neurovasculature, introduces additional challenges due to the tortuosity of the anatomy and the reduced size of the vessels. The increased tortuosity of the neurovasculature requires that devices used be more flexible, and the reduced size of the vessels requires that devices and any accessories reduce their size to accommodate the smaller vessels. Due to the small and tortuous vessels of the neurovasculature, devices intended to be used in this part of the anatomy must be specially designed to function appropriately. Special considerations must also be given to the limited use of accessory devices as access of multiple devices simultaneously in the neurovascular may be difficult or impossible.

When stents are being delivered, accuracy of stent placement is critical to ensure that the stent is as effective as possible. In more tortuous anatomies, such as near bifurcations or aneurysms, the accuracy of stent placement is of paramount importance. Should the stent be placed inaccurately such that it extends beyond the intended vessel wall and into the open vessel, there is significant patient risk for thrombus formation. This risk increases in critical anatomies, such as the neurovasculature, where an improperly placed stent may increase the risk of stroke in the patient. In larger vessels, there are established techniques using accessory devices, such as additional guidewires or balloon catheters, which can improve the accuracy of stent placement. In the neurovasculature, the reduced vessel diameter and tortuosity either prevents the usage of these techniques or increases their risk and/or difficulty. Therefore, there is a distinct need for a stent and stent delivery system which would improve the accuracy of stent placement without the need for accessories or advanced techniques.

One method of providing additional stent placement accuracy is to allow the stent to be recaptured prior to complete deployment. This would provide the interventionalist an opportunity to reposition the stent if it does not deploy in the desired location.

U.S. Pat. No. 7,309,351 discloses a stent which can be recaptured prior to complete deployment. More specifically, U.S. Pat. No. 7,309,351 discloses a self-expanding stent where the ends of the stent are comprised of a strut surrounded by a coil. The delivery system is a coil of variable diameter where the diameter variation aligns with the stent strut coil diameter. The coiled end of the stent then interacts with the delivery wire to anchor the device in place until the end is unsheathed. However, the stent and stenting technology disclosed in U.S. Pat. No. 7,309,351 introduces two (2) undesirable design limitations. First, by design, the anchor member requires that at least one end of the stent has an attached coil, creating an inconsistency in the stent geometry, which may promote thrombus generation post implantation. Second, in order for the delivery system to be compatible with standard neurovascular accessories (e.g., microcatheter), the stent must have very thin struts to accommodate the additional bulk introduced by both, the coils on both the stent ends and the mating coil on the delivery system. The thin struts required to make this a viable solution have a severe negative impact on the radial strength of the stent, reducing the overall effectiveness during and after implantation. Although the stent disclosed in U.S. Pat. No. 7,309,351 permits recapture, it is not capable of allowing for stent repositioning while not sacrificing patient outcomes or introducing additional risk.

Therefore, there still remains a need for a stent and corresponding delivery system which are able to provide a repositionable, low-profile stent which can meet the functional requirements to effectively treat blood vessel disorders without sacrificing patient outcomes or introducing additional risk.

### SUMMARY

At least the above-discussed need is addressed and technical solutions are achieved in the art by various embodiments of the present invention. In some embodiments of the invention, a stent device comprises a stent and a stent delivery system. The stent includes a cylindrical portion forming a stent frame having an outer lattice network of a plurality of interconnecting segments being configured to exert a radial force against an inner wall of a blood vessel. The stent delivery system comprises a locking mechanism configured to engage with the proximal end of the stent frame when covered in a sheath or microcatheter, and a push wire. The locking mechanism includes a proximal retention disk and a distal retention disk, the distal retention disk having a plurality of slots configured to engage with an equal plurality of struts formed on a proximal end of the stent.

In some embodiments of the invention, the locking mechanism of the stent delivery system is further configured to engage with the plurality of struts of the stent when the stent is in a partially deployed state to permit the stent to be retracted into an undeployed state.

In some embodiments of the invention, a distance between a position of the distal retention disk and a position of the proximal retention disk corresponds to a length and a radius of a proximal apex of the stent frame.

In some embodiments of the invention, a size of each of the plurality of slots of the distal retention disk is larger than a size of each of the plurality of struts of the stent to permit the proximal end of the stent frame to sit within the plurality of slots.

In some embodiments of the invention, the locking mechanism of the stent delivery system is further configured to disengage with the plurality of struts of the stent when the stent is in a fully deployed state to permit the stent to be placed in situ.

In some embodiments of the invention, the proximal end of the stent frame has an open geometry that permits a larger angle on the proximal apex when in a fully deployed state than in the undeployed state. In some embodiments of the invention, the proximal end of the stent frame includes two parallel struts connected by a radiused end while in the crimped state. In some embodiments of the invention, an angle of the proximal apex on the proximal end of stent frame is sufficiently large in the deployed state to cause the proximal end of the stent to angle radially inward towards a central axis of the cylinder when in the undeployed state.

In some embodiments of the invention, the stent further includes at least one fluoroscopic element visible under fluoroscopic imaging. In some embodiments of the invention, at least one of the distal retention disk or the proximal retention disk includes at least one fluoroscopic element visible under fluoroscopic imaging.

These and other embodiments of the invention are discussed in detail below.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 depicts a view of a stent delivery system according to an embodiment of the invention.

FIG. 2 depicts a side view of a stent and stent delivery system in the undeployed state, according to an embodiment of the invention.

FIG. 3 depicts a top view of a stent and stent delivery system in the undeployed state, according to an embodiment of the invention.

FIG. 4 depicts a side view of a stent and stent delivery system sheathed within a tube in the undeployed state, according to an embodiment of the invention.

FIG. 5 depicts a side view of a stent and stent delivery system in a partially deployed state, where the introducer sheath or microcatheter has been partially retracted to expose the distal end of the stent but not the proximal end of the stent, according to an embodiment of the invention.

FIG. 6 depicts the configuration from FIG. 5 without the introducer sheath or microcatheter in the view, according to an embodiment of the invention.

FIG. 7 depicts a side view of a stent and stent delivery system in a fully deployed state, according to an embodiment of the invention.

FIG. 8 depicts a top view of the configuration from FIG. 7, according to an embodiment of the invention.

FIG. 9 depicts a front view of a first distal retention disk, according to an embodiment of the invention.

FIG. 10 depicts a front view of a second distal retention disk, according to another embodiment of the invention.

FIG. 11 depicts a front view of a third distal retention disk, according to another embodiment of the invention.

FIG. 12 depicts a front view of a fourth distal retention disk, according to another embodiment of the invention.

FIG. 13 depicts a stent, according to an embodiment of the invention.

### DETAILED DESCRIPTION

In the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘including at least A’ includes A as well as the possibility of one or more other additional elements besides A. In the same manner, the phrase, ‘including A’ includes A, as well as the possibility of one or more other additional elements besides A. However, the phrase, ‘including only A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

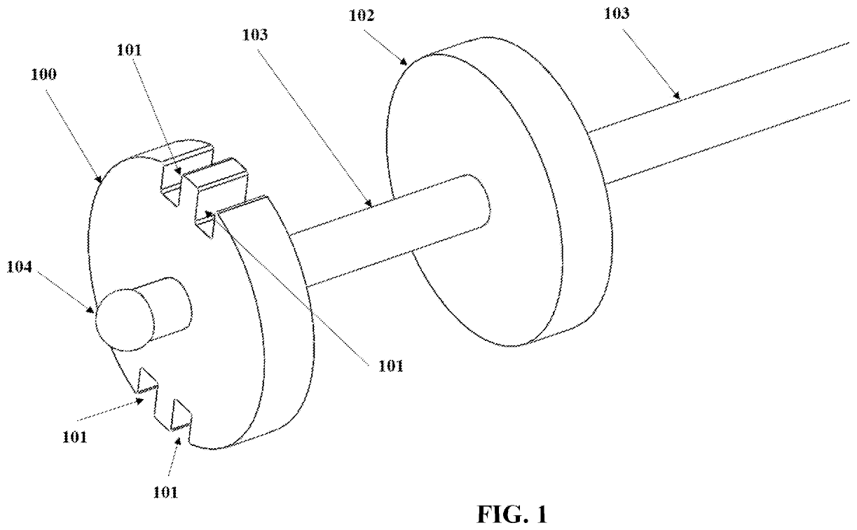

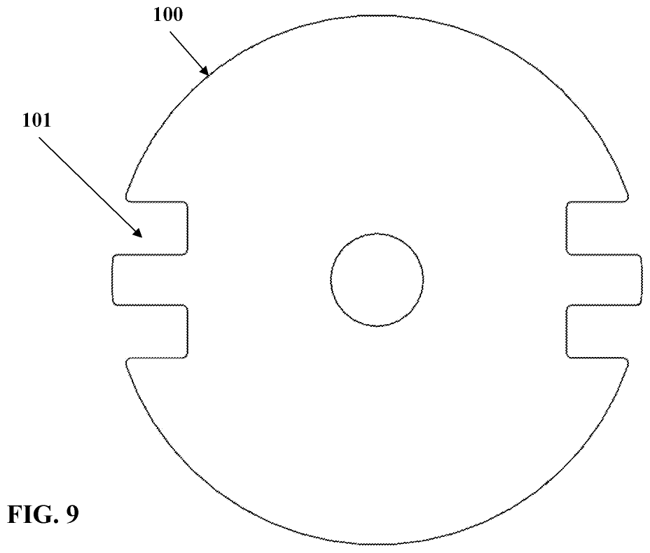

FIG. 1 shows a stent delivery system according to some embodiments of the present invention. The stent delivery system includes a distal retention disk 100 with four (4) distal retention disk slots 101 mounted to the distal end of a push wire 103. The stent delivery system also includes a proximal retention disk 102 mounted to the push wire 103 proximal to the distal retention disk 100. In some embodiments of the invention, the push wire 103 terminates to a push wire sphere 104 to create a gentler surface geometry and reduce the risk of damaging a patient's vessels during the procedure. In some embodiments of the invention, the push wire 103 may be a tapered wire. In some embodiments of the invention, the push wire 103 may be a coil or a combination of a wire and a coil to improve deliverability of the stent delivery system. In some embodiments of the invention, the distal retention disk 100 includes fewer or additional distal retention slots 101, as shown in FIGS. 9-12, to accommodate alternate stent designs or to provide additional retention force.

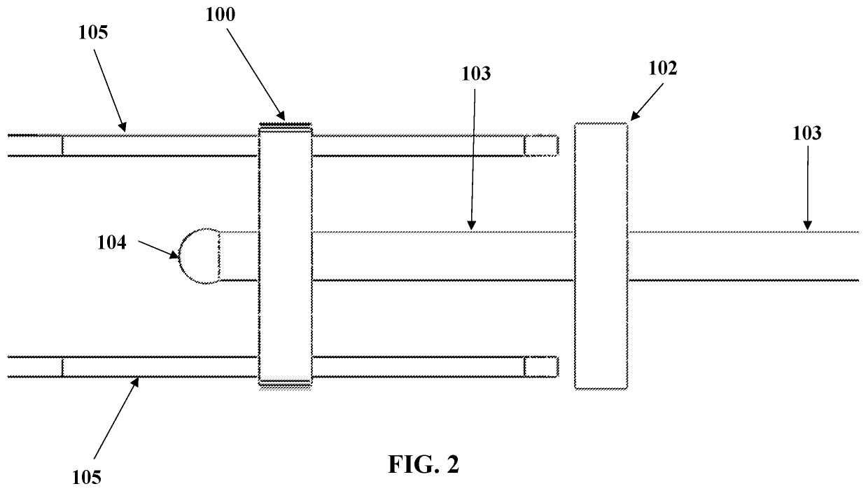

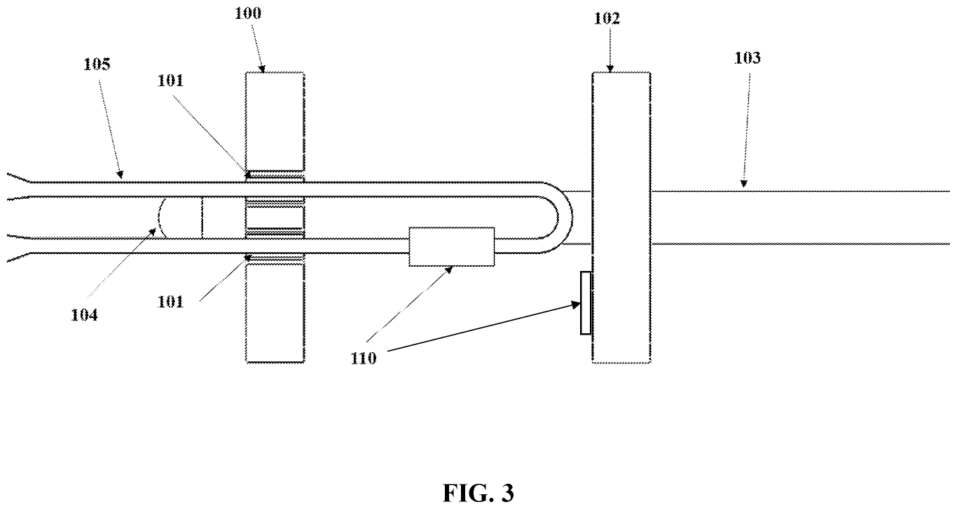

FIG. 2 and FIG. 3 show a stent delivery system loaded with a self-expanding stent 105 according to some embodiments of the present invention. FIG. 2 depicts a side view of the stent and stent delivery system in the undeployed state, where the stent is engaged within the delivery system locking mechanism (proximal and distal retention disks), according to an embodiment of the invention. FIG. 3 depicts a top view of the stent and stent delivery system in the undeployed state, where the stent is engaged within the delivery system locking mechanism (proximal and distal retention disks), according to an embodiment of the invention

The self-expanding stent 105 includes a plurality of interconnecting metallic segments forming a mesh tube. In some embodiments, the interconnecting metallic segments of the self-expanding stent 105 are formed of a superelastic metallic material, which allows the self-expanding stent 105 to expand without externally applied forces (e.g., a balloon catheter). In FIG. 2 and FIG. 3, the self-expanding stent 105 is in the undeployed, crimped, or sheathed configuration, with the self-expanding stent 105 fully engaged in the distal retention disk slots 101 and the entirety of the self-expanding stent 105 covered by the introducer sheath or microcatheter 106. In some embodiments, the distal retention disk slots 101 have a width slightly larger than that of the proximal end of the self-expanding stent 105. The disparity in the distal retention disk slot 101 size and the width of the proximal end of the self-expanding stent 105 allows the proximal end of the self-expanding stent 105 to sit inside of the distal retention disk slots 101. In some embodiments of the invention, the distal retention disk 100 includes four (4) distal retention disk slots 101, which provide channels for an equal number of self-expanding stent 105 struts 109. In some alternative embodiments of the invention, the distal retention disk 100 includes different numbers of distal retention disk slots 101 to accommodate alternative stent designs with different numbers of struts 109.

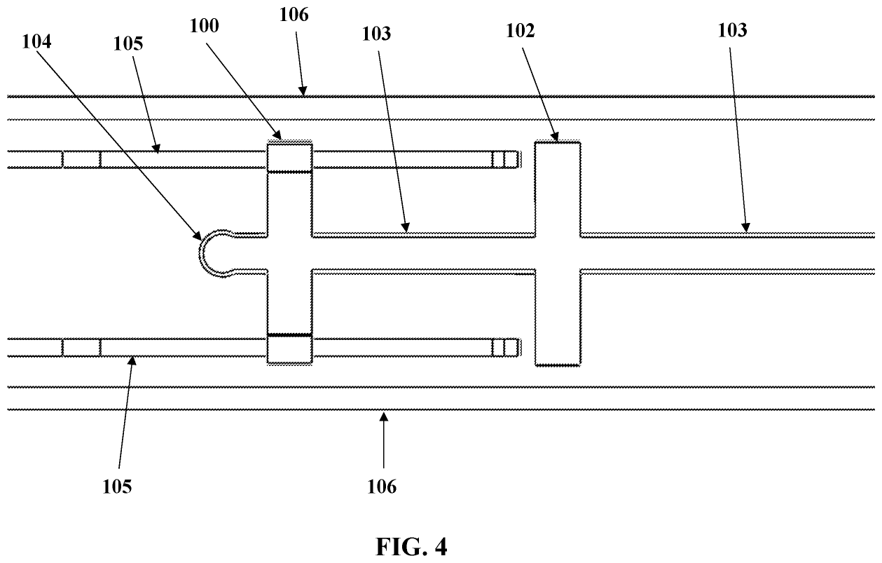

FIG. 4 depicts a side view of the stent and stent delivery system in the undeployed state, where the stent and stent delivery system are sheathed within a tube such as an introducer sheath or microcatheter 106, according to an embodiment of the invention. FIG. 4 demonstrates the same configuration of the stent as FIG. 2 but includes a microcatheter 106 or introducer sheath. In some embodiments of the invention, prior to use, the stent delivery system and self-expanding stent 105 may be loaded into an introducer sheath for packaging and transportation. In some embodiments of the invention, the introducer sheath may be comprised of a smooth plastic material that permits the stent delivery system and self-expanding stent 105 to travel along the inner diameter of the introducer sheath. In some embodiments of the invention, the introducer sheath has an inner diameter that is at least equal to the maximum diameter of the distal retention disk 100 and proximal retention disk 102 but no greater than the sum of the maximum diameters of the distal retention disk 100 and the thickness of the proximal strut 109 of the self-expanding stent 105. So long as the inner diameter of the introducer sheath aligns with the specification above, the proximal end of the self-expanding stent 105 will remain engaged in the distal retention disk slots 101.

Procedurally, in these embodiments where the stent 105 is packaged in the introducer sheath, the stent delivery system and self-expanding stent 105 is advanced from the introducer sheath to a microcatheter 106 which is used as the in-situ delivery accessory to allow the stent delivery system and self-expanding stent 105 to reach the target treatment location. In some embodiments of the invention, the microcatheter 106 has an inner diameter which is at least equal to the maximum diameter of the distal retention disk 100 and proximal retention disk 102 but no greater than sum of the maximum diameters of the distal retention disk 100 and the thickness of the proximal strut 109 of the self-expanding stent 105. So long as the inner diameter of the microcatheter 106 aligns with the specification above, the proximal end of the self-expanding stent 105 will remain engaged in the distal retention disk slots 101. In some embodiments, the self-expanding stent 105 may be packaged in the microcatheter 106 rather than the introducer sheath.

When the stent delivery system and self-expanding stent 105 are advanced through the microcatheter 106 and reach the target treatment location, the stent will be deployed by the interventionalist. The interventionalist will determine when it is appropriate to deploy the self-expanding stent 105 through the use of fluoroscopic imaging. In some embodiments of the invention, the self-expanding stent 105 contains one or more elements 110 that are visible under fluoroscopy. In some embodiments of the invention, the distal retention disk 100 and proximal retention disk 102 contain one or more elements 110 that are visible under fluoroscopy. In some embodiments of the invention, the stent delivery system contains elements that are visible under fluoroscopy. Deployment of a self-expanding stent 105 involves the unsheathing of the self-expanding stent 105. Unsheathing is done by retracting the introducer sheath or microcatheter 106 without moving the self-expanding stent 105 simultaneously, thereby exposing the self-expanding stent 105 from beneath the introducer sheath or microcatheter 106. By unsheathing the self-expanding stent 105, the diameter of the self-expanding stent 105 increases until it has reached its intended diameter. When the interventionalist is confident in the location of the stent delivery system and self-expanding stent 105 in relation to the intended treatment site, the interventionalist will retract the microcatheter 106 completely to reveal the entirety of the self-expanding stent 105, beginning with the distal end of the self-expanding stent 105. As the microcatheter 106 is being retracted, the diameter of the self-expanding stent 105 will increase. However, until the proximal end of the self-expanding stent 105 is unsheathed, the self-expanding stent 105 will remain engaged within the distal retention disk slots 101.

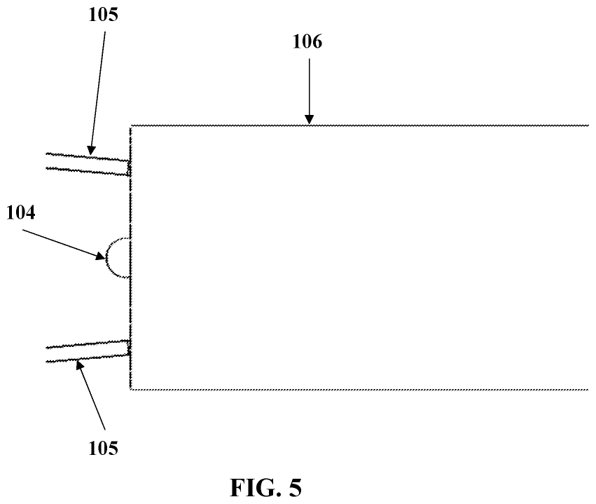

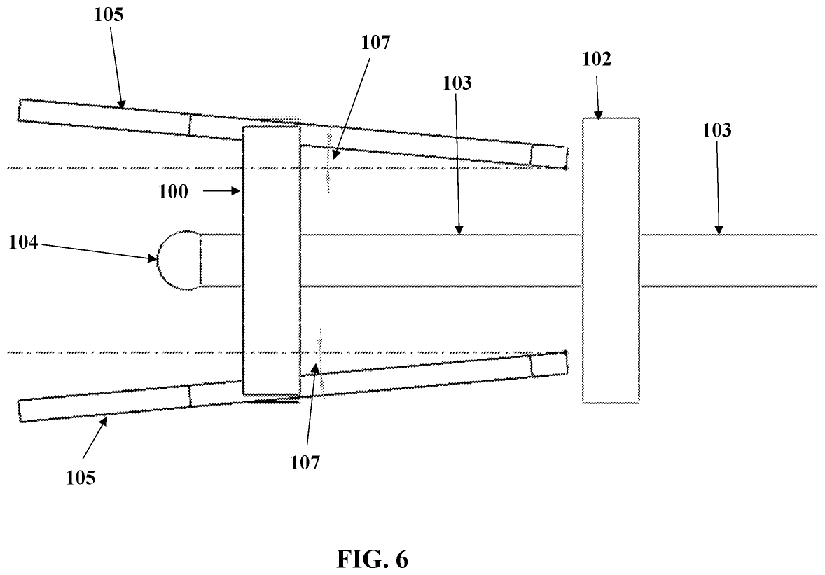

As the microcatheter 106 is retracted, the self-expanding stent 105 enters a partially deployed state, as shown in FIGS. 5 and 6. While in this partially deployed state, the proximal end of the self-expanding stent 105 remains engaged within the distal retention disk 100 and proximal retention disk 102. In some embodiments of the invention, the engagement force that engages the self-expanding stent 105 to the proximal end of the self-expanding stent 105 in the partially deployed state is enhanced as the partial deployment generates a stent frame radial angle 107, which further prevents the proximal end of the self-expanding stent 105 from becoming disengaged in the distal retention disk slots 101 by applying additional force against the disk slots 101.

During the partial deployment stage of the procedure, the interventionalist has the option to recapture or re-sheath the self-expanding stent 105 by advancing the microcatheter 106 over the distal portion of the stent. So long as the proximal end of the self-expanding stent 105 remains engaged with the distal retention disk 100 and proximal retention disk 102, the self-expanding stent 105 is able to be recaptured. The interventionalist may do this in order to reposition the stent should the initial deployment location not satisfy treatment requirements.

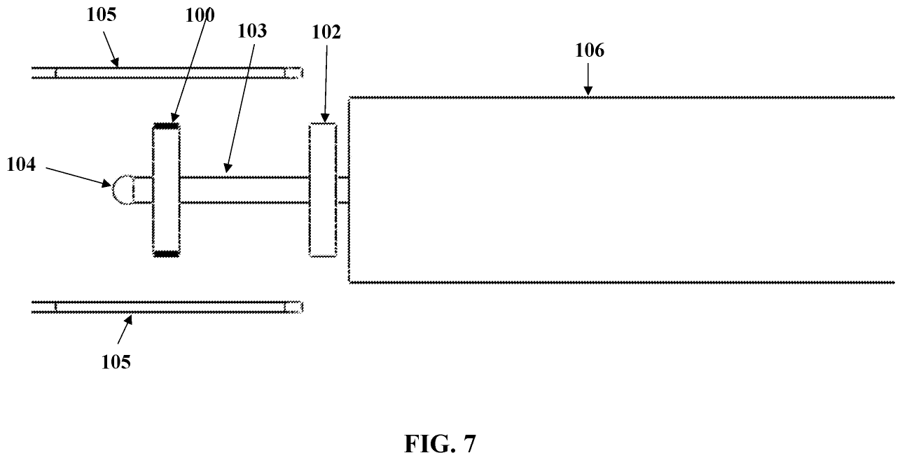

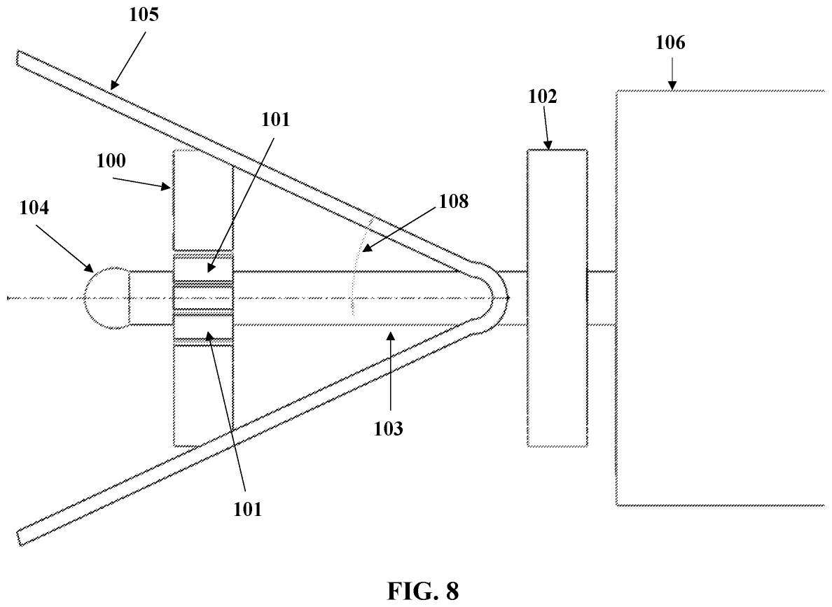

Once the interventionalist has confirmed proper location of the self-expanding stent 105 through partial deployment, the self-expanding stent 105 is then fully deployed by fully retracting the microcatheter 106, thereby exposing the proximal retention disk 102 and the most proximal end of the self-expanding stent 105, as shown in FIGS. 7 and 8. FIG. 7 depicts a side view of the stent and stent delivery system in the fully deployed state, where the microcatheter 106 has been fully retracted to expose the proximal retention disk 102 and the proximal end of the stent 105, and where the stent 105 has fully expanded, according to an embodiment of the invention. FIG. 8 depicts a top view of the configuration from FIG. 7. Thus, FIG. 8 depicts the stent and stent delivery system in the fully deployed state, where the microcatheter 106 has been fully retracted to expose the proximal retention disk 102 and the proximal end of the stent 105, allowing the stent 105 to open to its intended diameter. FIG. 8 also depicts the deployed angle of the proximal stent apex, according to an embodiment of the invention.

The push wire 103 and corresponding stent delivery system may then be completely removed from the patient along with the microcatheter 106, leaving the self-expanding stent 105 to remain in the target vessel as a permanent implant. When fully deployed, the self-expanding stent 105 supports the target vessel to treat blood vessel disorders or support supplemental devices for more complex treatments.

In some embodiments of the invention, the proximal end of the self-expanding stent 105 is laser cut from an open geometry with a stent frame apex angle 108, as shown in FIG. 8. The stent frame apex angle 108 is sufficiently large in the deployed configuration such that, when in the undeployed, or crimped, configuration, the proximal end of the self-expanding stent 105 is at a stent frame radial angle 107 capable of increasing the engagement force of the self-expanding stent 105 in the distal retention disk 100. This feature is increasingly important during the transfer of the stent delivery system and self-expanding stent 105 from the introducer sheath to the microcatheter 106, as differences in the inner diameters of the introducer sheath and the microcatheter 106 have the potential to cause disengagement of the self-expanding stent from the distal retention disk. In some embodiments of the invention, the stent frame apex angle 108 provides additional outward force on the distal retention disk 100 when in the undeployed state, further increasing stent retention in the delivery system locking mechanism. In some embodiments of the invention, the stent frame apex angle 108 promotes the release of the self-expanding stent 105 when the microcatheter 106 is retracted beyond the proximal end of the self-expanding stent 105. In this embodiment, when the compressive forces applied by the microcatheter 106 are removed, the stent frame apex angle 108 serves to force disengagement of the proximal end of the stent 105 from the delivery system locking mechanism.

In some embodiments of the invention, the distal retention disk 100 and the proximal retention disk 102 are spaced apart a sufficient amount to allow the proximal end of the self-expanding stent 105 to achieve the stent frame radial angle 107 previously described. In another embodiment of the invention, the proximal retention disk 102 provides additional stent retention while the device is being advanced within the microcatheter 106. While the stent 105 is under compressive forces caused by advancement through the microcatheter 106, the proximal retention disk 102 will further promote the stent frame radial angle 107, further increasing stent retention, especially in critical moments such as the transfer from the introducer sheath to the microcatheter 106.

FIGS. 9-12 depict front views of various distal retention disks 100, according to different embodiments of the invention. FIG. 9 depicts a front view of a first distal retention disk, showcasing distal retention disk grooves which mate with the proximal stent strut 109 in prior figures, according to an embodiment of the invention. In the embodiment shown in FIG. 9, the stent 105 has four (4) struts 109 that engage with the four (4) slots 101 of the retention disk 100.

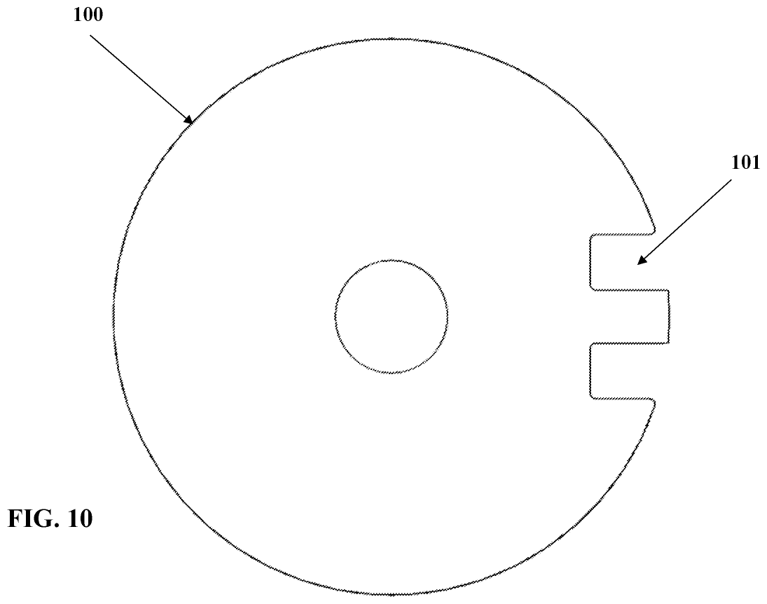

FIG. 10 depicts a front view of a second distal retention disk, according to another embodiment of the invention disk in a separate embodiment of the invention, where there are only two (2) slots 101 present for the stent delivery system locking mechanism. In the embodiment shown in FIG. 10, the stent 105 has two (2) struts 109 that engage with the two (2) slots 101 of the retention disk 100.

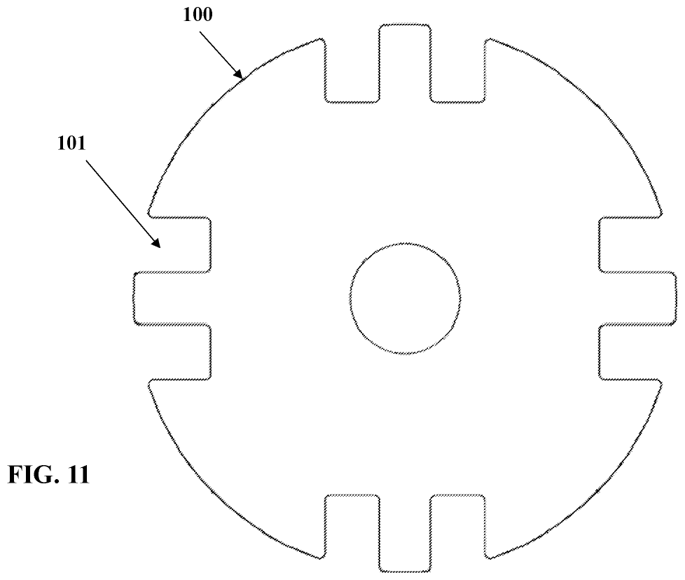

FIG. 11 depicts a front view of a third distal retention disk, according to another embodiment of the invention, where there are eight (8) slots 101 present for the stent delivery system locking mechanism. In the embodiment shown in FIG. 11, the stent 105 has eight (8) struts 109 that engage with the eight (8) slots 101 of the retention disk 100.

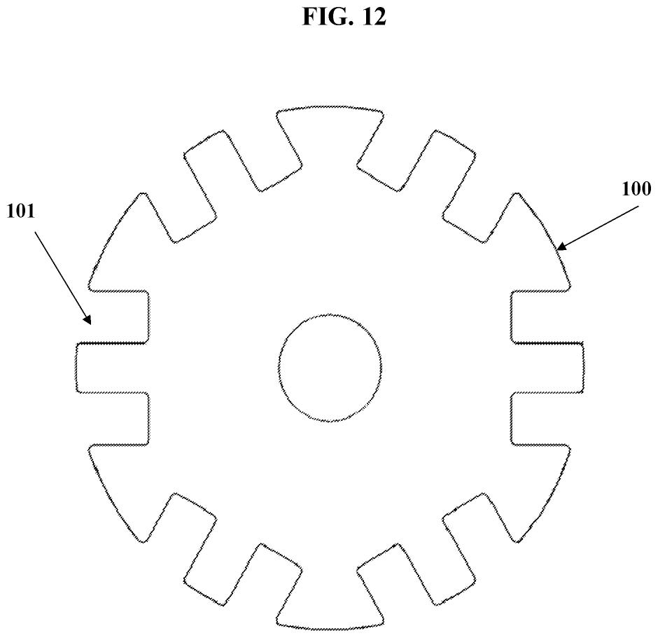

FIG. 12 depicts a front view of a fourth distal retention disk, according to another embodiment of the invention, where there are twelve (12) slots 101 present for the stent delivery system locking mechanism. In the embodiment shown in FIG. 12, the stent 105 has twelve (12) struts 109 that engage with the twelve (12) slots 101 of the retention disk 100.

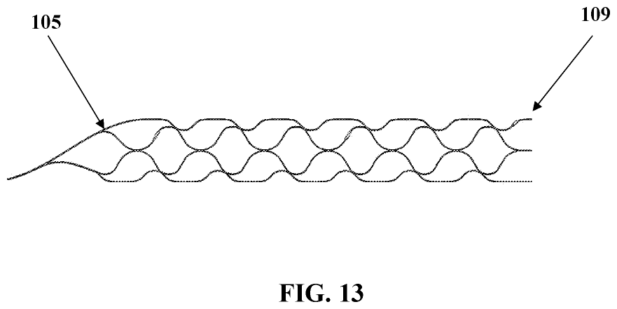

In some embodiments of the invention, a stent device comprises a stent 105 and a stent delivery system. As shown in the exemplar embodiment of FIG. 13, the stent 105 includes a cylindrical portion forming a stent frame having an outer lattice network of a plurality of interconnecting segments being configured to exert a radial force against an inner wall of a blood vessel. The stent delivery system comprises a locking mechanism configured to engage with the proximal end of the stent frame when covered in a sheath or microcatheter 106, and a push wire 103. The locking mechanism includes a proximal retention disk 102 and a distal retention disk 101, the distal retention disk 100 having a plurality of slots 101 configured to engage with an equal plurality of struts 109 formed on a proximal end of the stent 105.

In some embodiments of the invention, the locking mechanism of the stent delivery system is further configured to engage with the plurality of struts 109 of the stent 105 when the stent 105 is in a partially deployed state to permit the stent 105 to be retracted into an undeployed state.

In some embodiments of the invention, a distance between a position of the distal retention disk 100 and a position of the proximal retention disk 102 corresponds to a length and a radius of a proximal apex of the stent frame.

In some embodiments of the invention, a size of each of the plurality of slots 101 of the distal retention disk is larger than a size of each of the plurality of struts 109 of the stent 105 to permit the proximal end of the stent frame 105 to sit within the plurality of slots 101.

In some embodiments of the invention, the locking mechanism of the stent delivery system is further configured to disengage with the plurality of struts 109 of the stent 105 when the stent 105 is in a fully deployed state to permit the stent 105 to be placed in situ.

In some embodiments of the invention, the proximal end of the stent frame has an open geometry that permits a larger angle on the proximal apex when in a fully deployed state than in the undeployed state. In some embodiments of the invention, the proximal end of the stent frame includes two parallel struts 109 connected by a radiused end while in the crimped state. In some embodiments of the invention, an angle of the proximal apex on the proximal end of stent frame is sufficiently large in the deployed state to cause the proximal end of the stent 105 to angle radially inward towards a central axis of the cylinder when in the undeployed state.

In some embodiments of the invention, the stent 105 further includes at least one fluoroscopic element 110 visible under fluoroscopic imaging. In some embodiments of the invention, at least one of the distal retention disk 100 or the proximal retention disk 102 includes at least one fluoroscopic element 110 visible under fluoroscopic imaging.

It should be understood that the invention is not limited to the embodiments discussed above, which are provided for purposes of illustration only. Subsets or combinations of various embodiments described above provide further embodiments of the invention.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. A stent device comprising:
a stent including a cylindrical portion forming a stent frame having an outer lattice network of a plurality of interconnecting segments being configured to exert a radial force against an inner wall of a blood vessel; and
a stent delivery system comprising a locking mechanism configured to engage with the proximal end of the stent frame when covered in a sheath, and a push wire,
wherein the locking mechanism includes a proximal retention disk and a distal retention disk, the distal retention disk having a plurality of slots configured to engage with an equal plurality of struts formed on a proximal end of the stent.

2. The device according to claim 1, wherein the locking mechanism of the stent delivery system is further configured to engage with the plurality of struts of the stent when the stent is in a partially deployed state to permit the stent to be retracted into an undeployed state.

3. The device according to claim 1, wherein a distance between a position of the distal retention disk and a position of the proximal retention disk corresponds to a length and a radius of a proximal apex of the stent frame.

4. The device according to claim 1, wherein a size of each of the plurality of slots of the distal retention disk is larger than a size of each of the plurality of struts of the stent to permit the proximal end of the stent frame to sit within the plurality of slots.

5. The device according to claim 1, wherein the locking mechanism of the stent delivery system is further configured to disengage with the plurality of struts of the stent when the stent is in a fully deployed state to permit the stent to be placed in situ.

6. The device according to claim 1, wherein the proximal end of the stent frame has an open geometry that permits a larger angle on the proximal apex when in a fully deployed state than in the undeployed state.

7. The device according to claim 6, wherein the proximal end of the stent frame includes two parallel struts connected by a radiused end while in the crimped state.

8. The device according to claim 6, wherein an angle of the proximal apex on the proximal end of stent frame is sufficiently large in the deployed state to cause the proximal end of the stent to angle radially inward towards a central axis of the cylinder when in the undeployed state.

9. The device according to claim 1, wherein the stent further includes at least one fluoroscopic element visible under fluoroscopic imaging.

10. The device according to claim 1, wherein at least one of the distal retention disk or the proximal retention disk includes at least one fluoroscopic element visible under fluoroscopic imaging.
